(19) United States
(12) Patent Application Publication
(10) Pub. No.: US 2002/0072502 A1
(43) Pub. Date: Jun. 13, 2002

(54) Novel synthetic gangliosides

(75) Inventor: Tony W. Ho, Berwyn, PA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Neuronyx, Inc.

(21) Appl. No.: 09/945,346

(22) Filed: Aug. 31, 2001

Related U.S. Application Data
(60) Provisional application No. 60/229,883, filed on Sep. 1, 2000.

(51) Int. Cl. A61K 31/7028; C07H 15/00

(52) U.S. Cl. 514/23; 536/17.1; 536/116

(57) ABSTRACT

Disclosed is a synthetic ganglioside comprising a deamino-(2-O-substituted)-sphingosine group. Preferably, the deamino-(2-O-substituted)-sphingosine group is represented by Structural Formula (I):

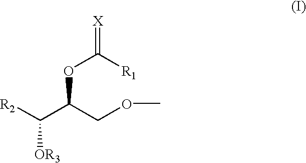

X is ═O or —H₂.

R₁ and R₂ are independently a substituted or unsubstituted straight chain or branched hydrocarbyl group, wherein the hydrocarbyl group optionally comprises —S—, —S(O)—, —SO₂—, —O— —NHCO—, —CONH—, —C(O)O—, —OC(O)—or —NR—.

R₃ is —H, —OS(O)₂OH, —OP(O)₂OH, —OP(O)₂OP(O)₂OH, —ON(O)OH.

Each R is independently —H, an aliphatic group, a substituted aliphatic group, an aryl group or a substituted aryl group.

Also disclosed are methods of treating a subject with a neurological condition or disease and methods of treating a subject in need of immunosuppresion. The methods comprises the step of administering to the subject an effective amount of the synthetic ganglioside represented by Structural Formula (I).

### RELATED APPLICATION

This application claims the benefit of U.S. Provisional Application No. 60/229,883, filed on Sep. 1, 2000. The entire teachings of the this application is incorporated herein by reference.

### BACKGROUND OF THE INVENTION

Neurological diseases and conditions (e.g., Alzheimer's disease, Parkinson's disease, stroke, amyotrophic lateral sclerosis) and autoimmune disorders (e.g., multiple sclerosis, rheumatoid arthritis, chronic polyarthritis, lupus erythematosus, juvenile-onset diabetes mellitus) can be progressive and debilitating. Certain neurological diseases and autoimmune disorders can ultimately result in the death of the affected subject.

Gangliosides have been used to treat some neurological diseases and autoimmune disorders. However, currently available treatment methods employing gangliosides are inadequate. Generally gangliosides used for therapeutic purposes have been purified by time consuming techniques from bovine brains resulting in potentially impure preparations. The gangliosides generally require intravenous administration because of insufficient absorption by the intestinal tract. Additionally, the currently available ganglioside have restricted passage through the blood brain barrier.

Thus, currently available gangliosides generally can not be readily prepared and conveniently administered to subjects to halt the progression, reduce the severity and/or treat neurological and autoimmune diseases and to promote neuritogenesis and neurogenesis. Thus, there is a need to develop new, improved and effective gangliosides for the treatment of neurological and autoimmune disease.

### SUMMARY OF THE INVENTION

The present invention is directed to novel synthetic gangliosides and methods of using said gangliosides for treating a subject with neurological conditions and diseases and for treating a subject in need of immunosuppression.

One embodiment of the present invention is a synthetic ganglioside comprising a deamino-(2-O-substituted)- sphingosine group. Preferably, the deamino-(2-O-substituted)-sphingosine group is represented by Structural Formula (I):

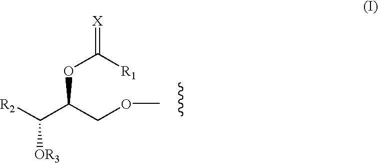

X is ═O or —H₂.

R₁ and R₂ are independently a substituted or unsubstituted straight chain or branched hydrocarbyl group, wherein the hydrocarbyl group optionally comprises —S—, —S(O)—, —SO₂—, —O— —NHCO—, —CONH—, —C(O)O—, —OC(O)—or —NR—.

R₃ is —H, —OS(O)₂OH, —OP(O)₂OH, —OP(O)₂OP(O)₂OH, —ON(O)OH. Preferably, R₃ is —H.

Each R is independently —H, an aliphatic group, a substituted aliphatic group, an aryl group or a substituted aryl group.

Another embodiment of the present invention is a method of treating a subject with a neurological disease or condition. The subject can be, for example, in need of neuroprotection, in need of neurogenesis or in need of neuritogenesis. The method comprises the step of administering to the subject an effective amount of the synthetic ganglioside represented by Structural Formula (I).

Yet another embodiment of the present invention is a method of treating a subject in need of immunosuppression, e.g., a subject with an organ, bone marrow or stem cell transplant or a subject with an autoimmune disease. The method comprises the step of administering to the subject an effective amount of the synthetic ganglioside represented by Structural Formula (I).

### BRIEF DESCRIPTION OF THE DRAWINGS

FIG. 1 is a schematic showing the synthesis of synthetic gangliosides of the present invention. The synthetic gangliosides comprise a deamino (2-O-substituted) sphingosine represented by Structural Formula (1), wherein X is ═O.

FIG. 2 is a structural formula depicting the structure of the ganglioside GM1.

FIG. 3 is a structural formula depicting the structure of the ganglioside GM2.

FIG. 4 is a structural formula depicting the structure of the ganglioside GD 1b.

FIG. 5 is a structural formula depicting the structure of the ganglioside GT1b.

FIG. 6 is a structural formula depicting the structure of the ganglioside GD2.

FIG. 7 is a structural formula depicting the structure of the ganglioside GM3.

FIG. 8 is a structural formula depicting the structure of the ganglioside GD3.

FIG. 9 is a schematic showing the synthesis of synthetic gangliosides with modified hydrocarbyl groups.

### DETAILED DESCRIPTION OF THE INVENTION

The compounds of the present invention are novel derivatives of a class of compounds known as gangliosides. The term “ganglioside” includes both naturally occurring and synthetic compounds and can be represented by Structural Formula (II):

A—B. (II)

A is an N-acylated sphingosine or modified sphingosine group; and B is a polysaccharide group. The synthetic compounds of the present invention differ from known gangliosides in the sphingosine portion of the ganglioside and comprise a polysaccharide portion from known naturally-occurring or synthetic gangliosides having either neuroprotective, neurogenic, neuritogenic or immunosuppresive activity.

The polysaccharide portion of naturally-occurring gangliosides comprises an oligosaccharide substituted with one or more and typically no more than five sialic acid units. A wide variety of biologically active naturally-occurring and synthetic gangliosides with variations in the polysaccharide portion of the molecule are known. The polysaccharide in a synthetic ganglioside is referred to herein as a “synthetic sialic acid substituted oligosaccharide”. The polysaccharide in a naturally-occurring ganglioside is referred to herein as a “naturally-occurring sialic acid substituted oligosaccharide”. The term “sialic acid substituted oligosaccharide” includes both the oligosaccharide and the sialic acid residue(s).

The oligosaccharide portion of a ganglioside typically has up to 5 monosaccharides or derivatives thereof comprising an acylamino group. Hexoses and hexose derivatives comprising an acylamino group are preferred. Typically, at least one glucose or galactose molecule is present in the oligosaccharide; and the most frequent acylamino derivatives of the aforesaid sugars are N-acetylglucosamine and N-acetylgalactosamine. Sialic acids are acylated derivatives of neuraminic acid, represented below by Structural Formula (III):

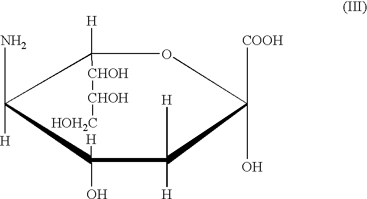

In naturally occurring gangliosides, the sialic acid groups are neuraminic acid residues in which the amine is acylated with acetic acid or glycolic acid. However, the present invention also encompasses biologically active synthetic ganglio sides in which sialic acid amine is acylated with other carboxylic acids, as described below. The number of sialic acids present in ganglio sides usually varies between 1 and 5. The sialic acid residues are generally connected to the oligosaccharide by a ketose bond formed from the hydroxyl group at the 2-position of the sialic acid residue and a hydroxyl group of the oligosaccharide. Alternatively, sialic residues can be connected to each other, typically by ketose bonds between the hydroxyls at positions 2 and 8 of two sialic acid molecules.

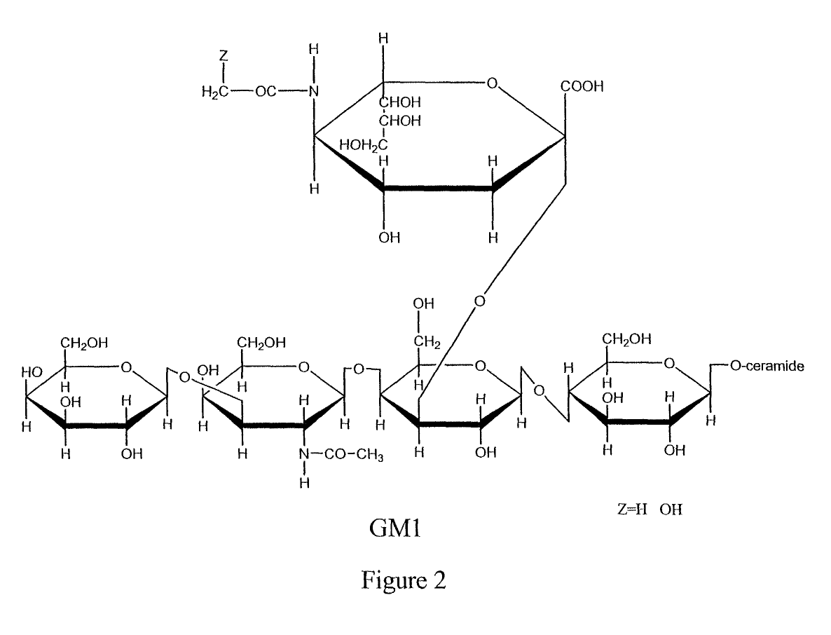

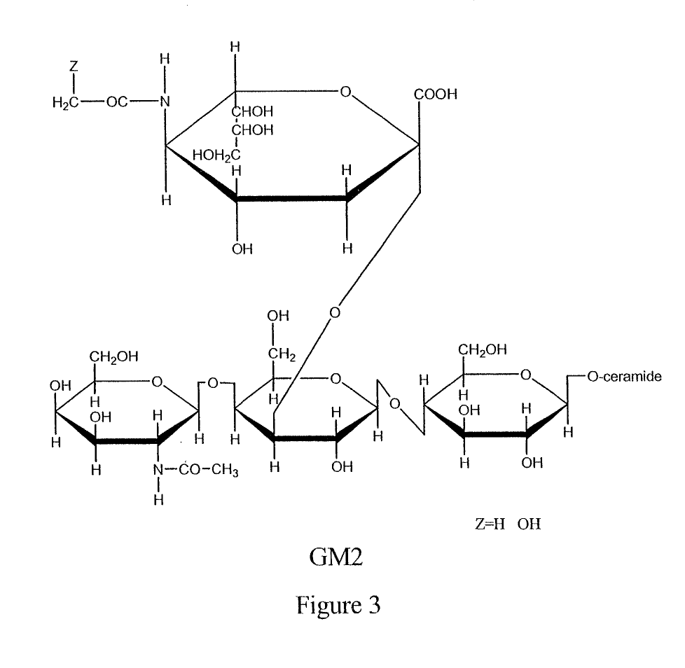

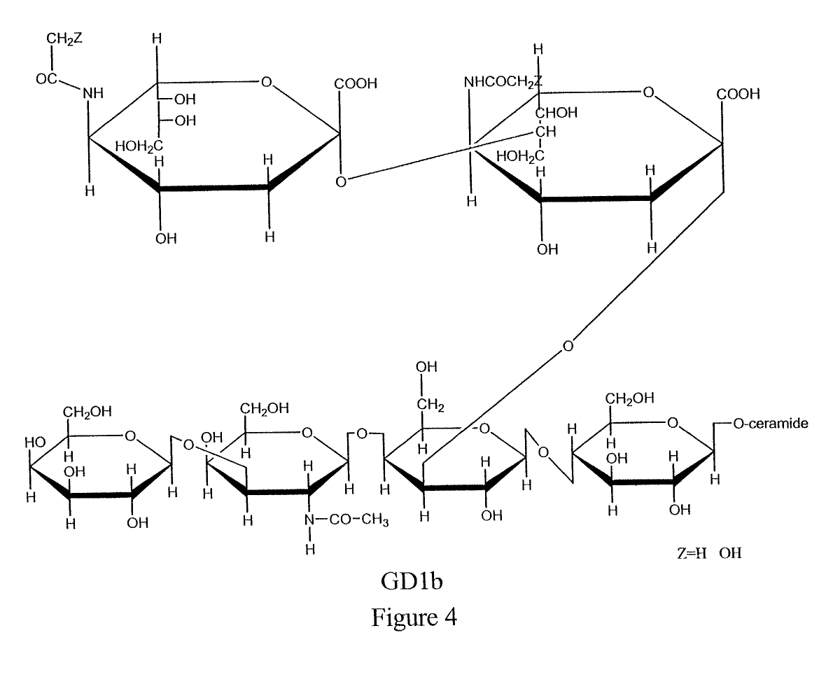

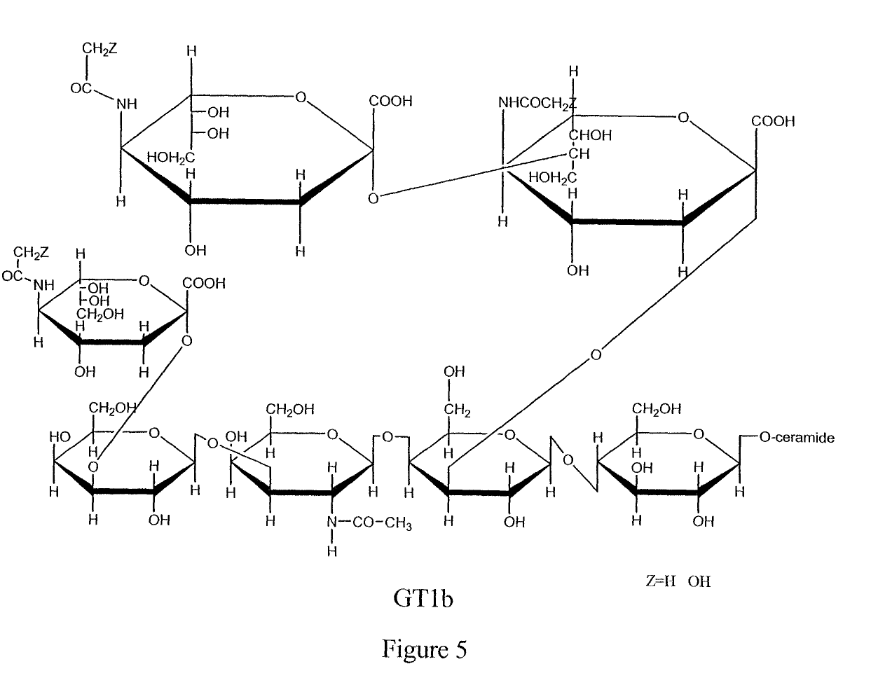

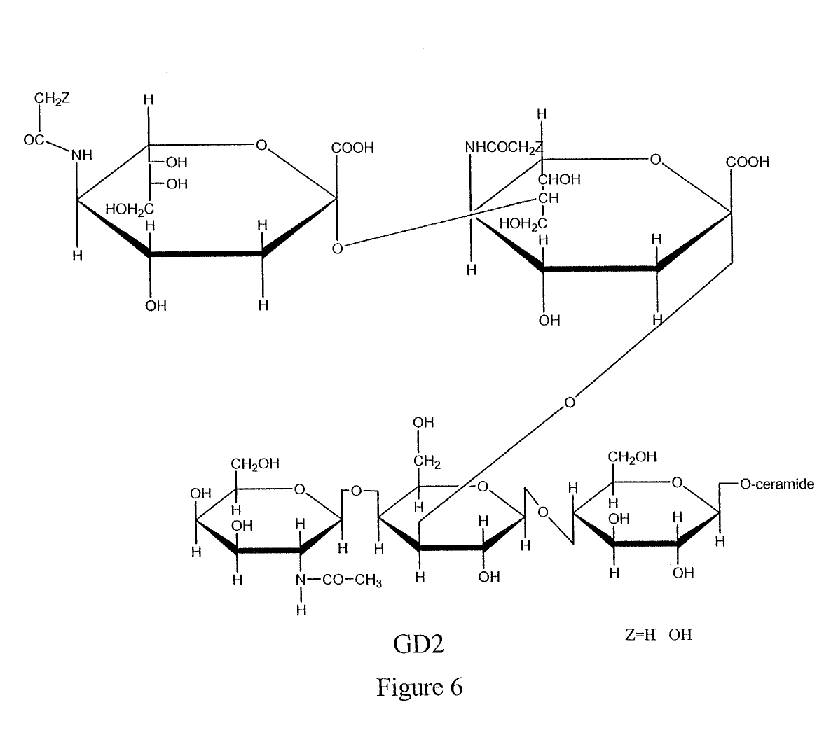

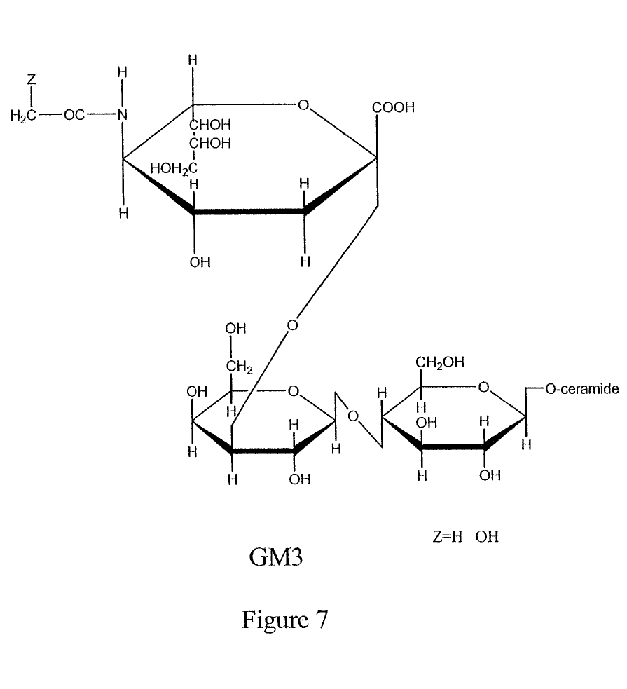

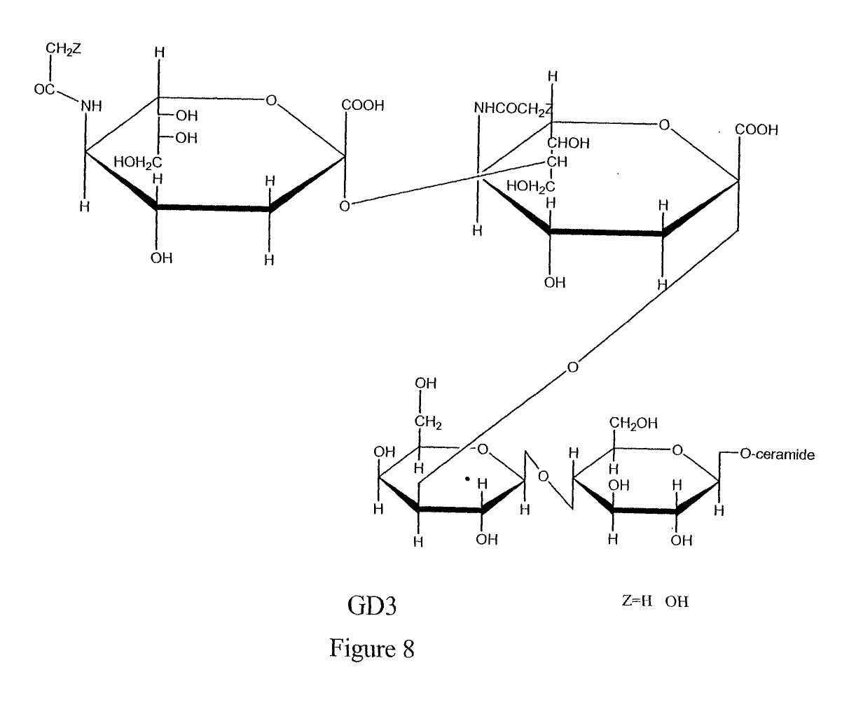

In one aspect, B in Structural Formula (II) is the sialic acid substituted oligosaccharride of a naturally occurring ganglioside. Examples include GM1, GM2, GD1b, GT1b , GD2, GM3, GD1a, GM1b, GT1a, GD3, GA2 and GA1. Preferred examples are the sialic acid substituted oligosaccharride of GM1, GM2, GD1b, GT1b, GD2, GM3 or GD3. The structures of GM1, GM2, GD1b, GT1b, GD2, GM3 and GD3 are shown in FIGS. 2-8. In another aspect, B in Structural Formula (II) is a synthetic sialic acid substituted oligosaccharride. Suitable synthetic sialic acid substituted oligosaccharides can be obtained by modifying the functional groups on naturally-occurring gangliosides, as described in greater detail below.

In one example of a synthetic ganglioside, the sialic acid substituted oligosaccharide is sulfated, i.e., one or more hydroxyl groups in the sialic acid substituted oligosaccharide is modified to form a sulfate ester. Synthetic gangliosides of this type are disclosed in U.S. Pat. No. 5,849,717.

In another example of a synthetic ganglioside, the carboxylic acid group in the sialic acid residue is esterified. Included are “inner esters,” i.e., where a lactone forms between the carboxyl group and a hydroxyl group in the oligosaccharide, and “outer esters”, i.e., where the carboxyl group is esterified with an alcohol RCOH. RC is an aliphatic group, a substituted aliphatic group, an aryl group or a substituted aryl group. Synthetic gangliosides of this type are disclosed in U.S. Pat. No. 5,264,424.

In another example of a synthetic ganglioside, the carboxylic acid group in the sialic acid residue is amidated with HNR^{a}R^{b} or with an aliphatic amino acid containing a carboxylic acid or sulfonic acid group. Rₐ and R_{b} are independently —H, an aliphatic group, a substituted aliphatic group, an aryl group or a substituted aryl group, or, taken together with the nitrogen atom to which they are bonded, a C2-C6 substituted or unsubstituted alkylene group. Synthetic gangliosides of this type are disclosed in U.S. Pat. No. 5,350,841.

In yet another example of a synthetic ganglioside, one or more of the hydroxyl groups in the oligosaccharide and/or sialic acid residue is acylated, i.e., is converted to —OCOR. R is as described above. Synthetic gangliosides of this type are disclosed in U.S. Pat. Nos. 5,484,775 and 5,264,424.

The entire teachings of the U.S. Pat. Nos. 5,849,717, 5,264,424, 5,350,841 and 5,484,775 are incorporated herein by reference. These references also teach methods of preparing the disclosed synthetic gangliosides.

Naturally occurring gangliosides generally comprise a ceramide group, which is shown below in Structural Formula (IV):

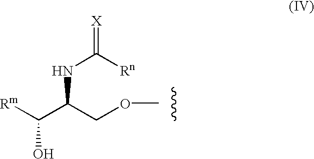

R^{m} is typically an alkyl or alkenyl group, whereas Rⁿ is typically an acyl group. A “sphingosine” group is a ceramide in which the acyl group has been removed from the amine at the two position. A “deamino sphingosine group” is a sphingosine group in which the amine at position two has been removed. A “deamino (2-O-substituted) sphingosine group” is a sphingosine group in which the amine at the two position has been replaced with a substituted alchohol, for example, an ether group (—OR) or an acetoxy group (—OCOR). The gangliosides of the present invention comprise a deamino (2-O-substituted) sphingosine group, one example of which is shown in Structural Formula (I).

An “aliphatic group” is non-aromatic, consists solely of carbon and hydrogen and may optionally contain or more units of unsaturation, e.g., double and/or triple bonds. An aliphatic group may be straight chained or branched and typically contains between about 1 and about 30 carbon atoms, more typically between about 1 and about 24 carbon atoms. Preferably, aliphatic groups are straight alkyl groups or straight chained alkenyl groups with one trans double bond.

A “hydrocarbyl group” is an aliphatic group which optionally contains a heteroatom containing functional group in place of a methylene, e.g., —S—, —S(O)—, —SO₂—, —O— —NHCO—, —CONH—, —C(O)O—, —OC(O)—or —NR—.

An “acyl group” is represented by —OCOR, where R is as described above.

Aromatic groups include carbocyclic aromatic groups such as phenyl, 1-naphthyl, 2-naphthyl, 1-anthracyl and 2-anthacyl, and heterocyclic aromatic groups such as N-imidazolyl, 2-imidazole, 2-thienyl, 3-thienyl, 2-faranyl, 3-furanyl, 2-pyridyl, 3-pyridyl, 4-pyridyl, 2-pyrimidy, 4-pyrimidyl, 2-pyranyl, 3-pyranyl, 3-pyrazolyl, 4-pyrazolyl, 5-pyrazolyl, 2-pyrazinyl, 2-thiazole, 4-thiazole, 5-thiazole, 2-oxazolyl, 4-oxazolyl and 5-oxazolyl.

Aromatic groups also include fused polycyclic aromatic ring systems in which a carbocyclic aromatic ring or heteroaryl ring is fused to one or more other heteroaryl rings. Examples include 2-benzothienyl, 3-benzothienyl, 2-benzofuranyl, 3-benzofuranyl, 2-indolyl, 3-indolyl, 2-quinolinyl, 3-quinolinyl, 2-benzothiazole, 2-benzooxazole, 2-benzimidazole, 2-quinolinyl, 3-quinolinyl, 1-isoquinolinyl, 3-quinolinyl, 1-isoindolyl and 3-isoindolyl.

Aliphatic, aryl and hydrocarbyl groups can be substituted with functional groups which do not significantly diminish the biological activity of the molecule. Examples include chloride, bromide, iodide, —OR, keto, ketal, acetal, ═O, —COR, —N═NR, —SR, aryl group, substituted aryl group, —COOR, —SO₃R, —SO₂NRₐR_{b}, —S(O)R, —SO₂R, —CN and —NRₐR_{b}. R, Rₐ, and R_{b} are as described above. In addition, aryl groups can be substituted with substituted or unsubstituted aliphatic groups; and hydrocarbyl and aliphatic groups can be substituted with substituted or unsubstituted aryl groups.

In a preferred embodiment of the present invention, A in Structural Formula (I) is a deamino (2-O-substituted) ganglioside, preferably represented by Structural Formula (I). In Structural Formula (I), R₁ and R₂ are preferably independently a substituted or unsubstituted straight chain or branched aliphatic group and R₃ is —H. Examples of suitable substituents for aliphatic groups are described above. More preferably, R₁ and R₂ are independently a straight chain aliphatic group optionally substituted with one or more halide groups and R₃ is —H. In one aspect, one of R₁ or R₂ is a straight chain C1-C24 alkyl or alkenyl group optionally substituted with one or more halide groups (preferably unsubstituted or chlorinated C1, C2, C3, C4, C5, C6, C7 or C8, preferably C1-C2 alkyl group optionally substituted with one, two or three chloride groups) and the other is a —CH₂CH₂—(CH₂),CH₃ or trans-CH═CH—(CH₂)ₙCH₃, wherein n is an integer from about nine to about 21 (e.g., C12, C13, C14, C15, C16, C17 or C18), preferably about ten to about fourteen. In specific examples, R₁ is —CHCl₂ and R₂ is trans-CH═CH—(CH₂)₁₂CH₃; and R₁ is —(CH₂)₁₄CH₃ and R₂ is —CHCl₂ In another aspect, R₁ and R₂ are independently a straight chain C1-C24 alkyl or alkenyl group optionally substituted with one or more halide groups (preferably unsubstituted or chlorinated C1, C2, C3, C4, C5, C6, C7, C8, C9, C10, C11, C12, C13, C14, C15, C16). Preferably at least one of R₁ or R₂ is a C1-C2 alkyl group optionally substituted with one, two or three chloride groups, such as —CHCl₂.

The O-acyl group (—OCOR,) at the 2-position of the modified sphingosine group represented by Structural Formula (1) when X is ═O can be derived from a wide variety of carboxylic acids (or corresponding acid halide). A non-limiting list includes: dichloroacetic acid, trichloroacetic acid and their fluorinated or brominated analogues; 2,2-dichloropropionic acid, 2,3-dichloropropionic acid, 2,2,3-trichloropropionic acid, normal-2,2-dichlorobutyric acid, 2,2-dichlorovalerianic acid, 2-chloroisovalerianic acid, 2,3-dichlorovalerianic acid, pentafluoropropionic acid, 3,3-dichloropivalic acid, 3-chloro-2,2-dimethylpropionic acid, chloro-difluoroacetic acid, 2,2-dichlorocapronic acid, 2-monochloropropionic, normal-2-monochlorobutyric, 2-monochlorovalerianic, and 2-monochlorocapronic acids and the fluorinated or brominated analogues of these acids; 2-chloropalmitic acid, 2-chlorostearic acid, 2-chlorooleic acid, 2-chlorolaurinic acid, 2-chlorobehenic acid, 4-chlorophenoxyacetic acid, 2-hydroxypropionic acid (lactic acid), 3-hydroxypropionic acid, 2-hydroxybutyric acid, 2-hydroxyvalerianic acid, 3-hydroxyvalerianic acid, 2,3-dihydroxybutyric acid and 2,3-dihydroxyvalerianic acid and C1-C4 lower aliphatic ethers or esters thereof; methoxyacetic acid, 12-hydroxystearic acid, 2-(4-hydroxyphenoxy) propionic acid, 2-hydroxyisocapronic acid, 2-hydroxyisobutyric acid and 4-fluoro-phenoxyacetic acid; pyruvic acid, acetacetic acid, levulinic acid and ketals thereof with lower aliphatic alcohols having a maximum of 4 carbon atoms; mercaptoacetic, 2-mercaptopropionic, 2-mercaptobutyric and 2-mercaptovalerianic acids and C1-C4 lower aliphatic thioethers or thioesters thereof; 2-mercaptolaurinic, oleic and palmitic acids and C1-C4 lower aliphatic thioethers or thioesters thereof; malonic acid, glutaric acid, monomethylglutaric acid, 3-hydroxy-3-methylglutaric acid, maleic acid, malic acid, succinic acid, fumaric acid, azelaic acid and C1-C4 aliphatic esters thereof; sulfoacetic acid, 2-sulfopropionic acid, 2-sulfobutyric acid, 2-sulfovalerianic acid and C1-C4 aliphatic sulfate esters thereof. Also included are 2-sulfolaurinic acid, 2-sulfooleic acid, 2-sulfopalmitic acid, 2-sulfostearic acid and C1-C4 lower aliphatic sulfate esters thereof; sulfamides or the sulfamides wherein the amine is optionally substituted with one or two C1-C4 lower alkyl groups or by C4-C6 alkylene groups; acetic acid, propionic, butyric and valerianic acids substituted in the 2-position by a C1-C4 alkyl, acylsulfoxide or C1-C4 alkylsulfone group; cyanacetic acid, 2-cyanpropionic acid, 2-cyanbutyric acid, 2-cyanvalerianic acid, aminoacetic acid, 2-aminopropionic acid, 2-aminobutyric acid, 3-aminobutyric acid, 4-aminobutyric acid, 2-aminovalerianic acid, 4-aminovalerianic acid and derivatives thereof with the amine optionally substituted with one or two C1-C4 alkyls, C4-C6 alkylene groups or C1-C4 acyl group; di-methylglycine, 3-diethylaminopropionic acid, camitine, and cysteic acid. Specific examples of synthetic gangliosides of the present invention are represented by Structural Formula (II), wherein B is the sialic acid substituted oligosaccharride of GM1, GM2, GD1b, GT1b, GD2, GD3 or GM3, A is represented by Structural Formula (I), X is ═O, the acylated alcohol R₁COO— is derived from one of the aforementioned carboxylic acids (or corresponding acid halide), R₂ is a straight chain C1-C24 alkyl or alkenyl group optionally substituted with one or more halide groups (preferably unsubstituted C10, C11, C12, C13, C14, C15, C16, C17 or C18), and R₃ is —H.

Other specific examples of synthetic gangliosides of the present invention are represented by Structural Formula (II), wherein B is the sialic acid substituted oligosaccharride of GM1, GM2, GD1b, GT1b, GD2, GD3 or GM3, A is represented by Structural Formula (I), X is ═H₂, R₁CH₂— corresponds to the alkyl portion of the carboxylic acids listed in the previous paragraph, R₂ is a straight chain C1-C24 alkyl or alkenyl group optionally substituted with one or more halide groups (preferably unsubstituted C10, C11, C12, C13, C14, C15, C16, C17 or C18) and R₃ is —H.

In addition, the aforementioned carboxylic acids are a non-limiting list of carboxylic acids from which acyl groups that modify the oligosaccharide and/or sialic acid residues in synthetic gangliosides can be derived.

In another embodiment, the deamino (2-O-substituted) sphingosine is represented by Structural Formula (V):

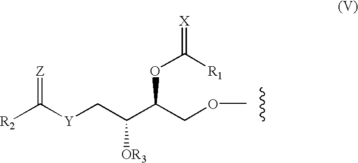

R₁, R₂, R₃ and X are as described above; Y is —NH— or —O—; and Z is ═O or H₂.

In the structural formulas depicted herein, the bond by which a chemical group or moiety is connected to the remainder of the molecule or compound is indicated by the following symbol:

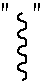

For example, the corresponding symbol in Structural Formula (I) indicates that the deamino (2-O-substituted) sphingosine group is connected to the sialic acid substituted oligosaccharide of the synthetic ganglioside alkylene group by a single covalent bond between the oxygen atom attached to the methylene carbon and an anomeric carbon atom of the oligosaccharide.

Also included in the present invention are pharmaceutically acceptable salts of the synthetic gangliosides described herein. Synthetic gangliosides of this invention which possess a sufficiently acidic, a sufficiently basic, or both functional groups, and accordingly can react with any of a number of inorganic bases, and inorganic and organic acids, to form a salt. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The compounds of the present invention are expected to be neuroprotective (e.g., protects neurons and glia), neurogenic (e.g., promotes differentiation of neurons and proliferation or differentiation of stem cells and progenitor cells) and/or neuritogenic (e.g., promotes neurite outgrowth and synaptogenesis) and are therefore expected to be useful to treat a wide variety of neurological diseases and conditions. For example, neuroprotective compounds are useful in treatment of the following degeneration diseases or lesions: ischemia, hypoxia, epilepsy, metabolic dysfunction, aging, toxic diseases and chronic neurodegeneration such as Alzheimer's disease, Amytropic Lateral Sclerosis, Parkinson's disease or Huntington's chorea. Neuritogenic compounds can advantageously be used, for example, in therapies aimed at nervous function recovery, such as in peripheral neuropathies and pathologies associated with neuronal damage (e.g., stroke, ischemic injuries, transverse myelitis, trauma, spinal cord injuries and neuropathies associated with diabetes).

The compounds of the present invention are also expected to inhibit proliferation of a number of different cell types of the immune system (e.g., CD₄⁺ T cells, lymphocytes and NK cells) and to inhibit the production of certain cytokines. Thus, they are expected to be immunosuppressive and therefore useful for the treatment and/or prevention of systemic or organ-specific autoimmune diseases, such as multiple sclerosis, rheumatoid arthritis, sarcoid, paraneoplastic disease, Sjögren, psoriasis, scleroderma, vasculitides, chronic polyarthritis, lupus erythematosus, juvenile-onset diabetes mellitus, and also to prevent organ transplant rejection as well as rejection by the transplanted material against the host, as in the case of bone marrow or stem cell transplant.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

An “effective amount” of a synthetic ganglioside is a quantity sufficient for neuroprotection, neurogenic, neuritogenic or immunosuppressive activity in a subject. Alternatively, an “effective amount” is a quantity sufficient to achieve a desired therapeutic and/or prophylactic effect, such as an amount which results in the prevention of or a decrease in the symptoms associated with a disease or condition for which ganglioside treatment is required, e.g., a neurogenerative disease or lesion, an autoimmune disease, nervous system degeneration, traumatic or ischemic nervous system injury or suppression of organ transplant rejection.

The amount of synthetic ganglioside administered to the individual will depend on the type and severity of the disease or condition and on the characteristics of the individual, such as general health, age, sex, body weight and tolerance to drugs. It will also depend on the degree, severity and type of disease or condition. The skilled artisan will be able to determine appropriate dosages depending on these and other factors. Typically, an effective amount of the synthetic gangliosidecan range from about 0.1 mg per day to about 1 gram per day for an adult. Preferably, the dosage ranges from about 1 mg per day to about 100 mg per day. A synthetic ganglioside can also be administered in combination with one or more additional therapeutic agents known to bring about a desired therapeutic effect for the disease or condition being treated.

The synthetic ganglioside can be administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The synthetic ganglioside can also be administered orally (e.g., dietary), topically, by inhalation (e.g., intrabronchial, intranasal, oral inhalation or intranasal drops), or rectally, depending on the disease or condition to be treated. Oral or parenteral administration are preferred modes of administration.

The synthetic ganglioside can be administered to the individual in conjunction with an acceptable pharmaceutical carrier as part of a pharmaceutical composition for treatment of a neurogenerative disease or lesion, an autoimmune disease, nervous system degeneration, nervous system injury or suppression of organ transplant rejection, or one of the other diseases discussed above. Formulation of a synthetic ganglioside to be administered will vary according to the route of administration selected (e.g., solution, emulsion, capsule). Suitable pharmaceutical carriers may contain inert ingredients which do not interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

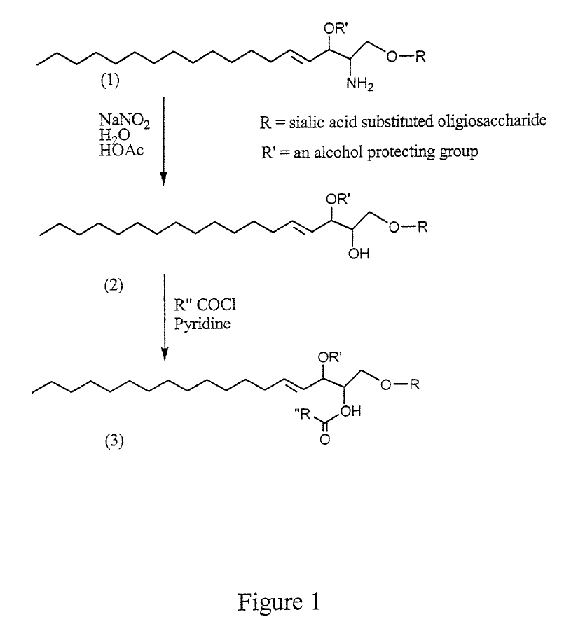

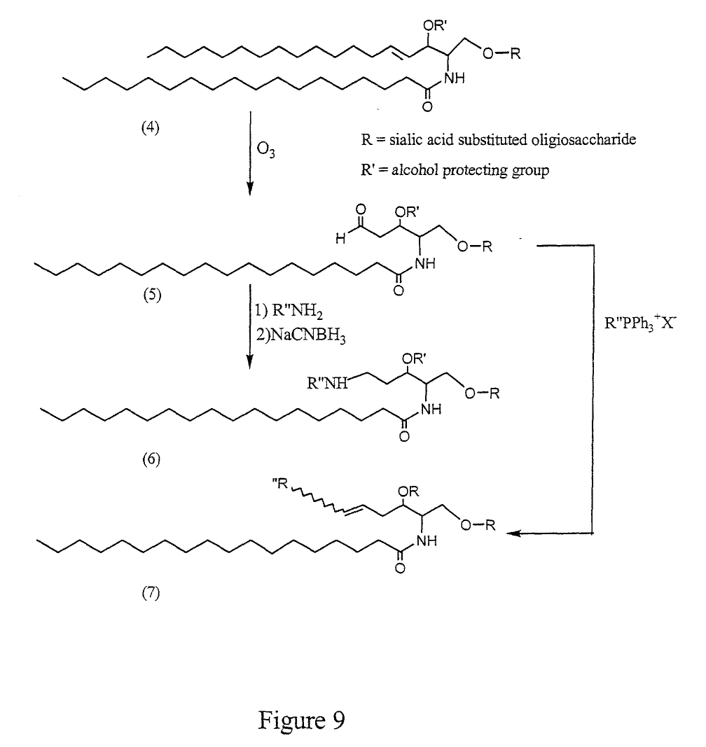

The synthetic gangliosides of the present invention can be prepared according to the synthetic schemes shown in FIGS. 1 and 9. The starting material (1) in FIG. 1 can be prepared by first selectively deacylating the amine at the two position in the sphingosine of a naturally occurring ganglioside, and protecting the free amine. These two reactions are specifically described in Neuenhofer et al., Biochemistry 24:525 (1985), the entire teachings of which are incorporated herein by reference. The alcohol group at the three position is then protected and the amine protecting group is removed. Suitable protecting groups and methods of applying and deprotecting are well known in the art and are described, for example, in Greene and Wuts, “Protective Groups in Organic Synthesis”, John Wiley & Sons (1991), the entire teachings of which are incorporated into this application by reference.

The starting material (1) in FIG. 1 is then diazotized in the presence of water to form compound (2). Conditions for carrying out this reaction are described in, for example, Shoppee and Sly, J Chem. Soc. 1959:345 (1959), the entire teachings of which are incorporated herein by reference. The free alcohol group at position two can then be acylated to form compound (3) in FIG. 1, whose structure corresponds to Structural Formula (I), wherein X is ═O. Alternatively, the free alcohol at position two can be etherified to a compound whose structure corresponds to Structural Formula (I), wherein X is —H₂ These reactions are well known in the art. For example, conditions for acylating a free alcohol are provided in March, “Advanced Organic Chemistry—Reactions, Mechanisms and Structure”, John Wiley & Sons, third edition, pages 346-47 and references cited therein; and conditions for etherifying a free alcohol are provided in March, supra, pages 342-43 and references cited therein. The entire teachings of these pages of March are incorporated herein by reference.

The reaction scheme shown in FIG. 9 provides a method of replacing the sphingosine hydrocarbyl group with another hydrocarbyl group of different length or a hydrocarbyl group comprising a heteroatom-containing functional group such as in Structural Formula (V). The double bond in the sphingosine portion of ganglioside (4) is ozonized to form aldehyde (5). The ozonolysis reaction is known in the art and specific conditions for carrying out this reaction are disclosed in Helling et el., Cancer Research 554:197 (1994), the entire teachings of which are incorporated herein by reference. Aldehyde (5) can then be reductively aminated with an amine R′NH₂ and sodium cyanoborohydride to form synthetic gangliosides (6). Reductive aminations are well known in the art and conditions for carrying out this reaction are taught, for example, in Lane, Sodium Cyanoborohydride: A Highly Selective Reducing Agent” in SELECTIONS FROM ALDRICHIMICA ACTA, Aldrich Chemcial Co., Inc. (1984), pages 67-78, and references cited therein. The entire teachings of this article are incorporated herein by reference. Specific procedures for carrying out this reductive amination are also disclosed in Helling et al., supra.

Alternatively, aldehyde (5) is reductively aminated with ammonia and sodium cyanoborohydride, thereby replacing —CHO with —CH₂NH₂. The primary amine is then acylated to form synthetic gangliosides represented by Structural Formula (V), wherein Z is ═O and Y is —NH—. Acylations of primary amines are well known in the art and specific conditions for carrying out this reaction are taught in U.S. Pat. No. 5,484,775, the entire teachings of which are incorporated herein by reference. Typically, the carboxylic acid and amine are reacted at room temperature in dimethylformamide in the presence of chloromethylpyridinium iodide.

In another alternative, aldehyde (5) is reduced with an aldehyde reducing agent such as sodium borohydride or lithium aluminum hydride to form an alcohol at position three, which can be etherified or esterified to form a ganglioside with a modified sphingosine represented by Structural Formula (V), wherein Z is —H₂ or ═O, respectively, and Y is —O—. Reactions for reducing aldehydes to alcohols are well known Ad in the art. For example, conditions for the reduction of aldehydes to alcohols are provided in March, “Advanced Organic Chemistry—Reactions, Mechanisms and Structure”, John Wiley & Sons, third edition, pages 809-14, the entire teachings of which are incorporated herein by reference. As noted above, reactions for etherifying and esterifying alcohols are also well known in the art.

Alternatively, aldehyde (5) is reacted with a Wittig reagent to add an alkenyl chain onto the aldehyde, thereby forming compound (7). Wittig reactions are well known in the art and are described in March, supra, pages 845-54. Optionally, the double bond can be hydrogenated according to known procedures. Protection of the alcohol group at position three of the modified sphingosine and in the polysaccharide may be required prior to the Wittig reaction, the reduction, or reductive animation. As noted above, suitable protecting are known in the art and are disclosed, for example, in Greene and Wuts, supra.

The biological activity of the compounds of the present invention can be evaluated by assays known in the art. For example, neuroprotective activity can be assessed by the assay described in Example 1; immunosuppressive activity can be evaluated by the assay described in Example 2; and neuritogenic activity can be evaluated by the assay described in Example 3.

### Exemplification

### EXAMPLE 1

### Neuroprotection Assay

I. Cell Sources

Four cell sources can be used for each of the assays. They are as follows:

SH-SY5Y (Human Dopaminergic Neuroblastoma Cells):

Human SH-SY5Y cells are cultured as described in Cassarino et al., Neurochem. 74: 1384 (2000). They are maintained in Dulbecco's modified Eagle's medium supplemented with 10% fetal calf serum and IX penicillin/streptomycin in T-75 flasks. Cells are grown to 80% confluence at 37° C. and 5% carbon dioxide, before starting drug/toxin treatment.

Primary Mesencephalic Cultures from Rat:

Pregnant Sprague Dawley rats at gestational day 15 (E 15) are used for preparing mesencephalic cultures (Dalman and O'Malley, J. Neurosci. 19:5750 (1999). Embryos are removed and the ventral mesencephalon are dissected out and placed in L-14 medium. The tissue is minced and incubated with 0.25% trypsin and 0.05% DNAase in PBS for 15 minutes at 37° C. The supernatant is replaced with DMEM with 10% fetal calf serum and the tissue aggregates dissociated by trituration with a constricted-bore glass pipette. The cell suspension are nylon filtered and transferred to Neurobasal medium (NB), B27 supplement, 0.5 mM glutamine, 100 U/ml penicillin and 100 μg/ml streptomycin and plated on to the prepared plates at 1.5×10⁵ cells/cm 2 in 500 μl of Neurobasal medium. At 3-day intervals the medium is refreshed by replacing one-half with fresh Neurobasal medium. These cells are then used for drug/toxin experiments.

Rat Cortical Neuronal Cultures:

Primary cortical neuronal cells are prepared from E-15 Sprague-Dawley rats as described (MacManus, et al., Exp. Cell Res. 233:310 (1997)). In brief, the dissociated cells are resuspended in minimum essential medium containing 10% fetal bovine serum and 10% heat-inactivated horse serum supplemented with 2 mM L-glutamine. Cells are seeded at a density of 10⁵/cm² in either 35-mm-diameter tissue culture dishes or 24-well plates. Ten micromolar cytosine arabinoside (Sigma) is added to the culture medium 18-22 hours after plating to eliminate the proliferation of non-neuronal cells. For microscopic observation, cells are plated on to 12 mm glass coverslips that have been precoated with poly-1-lysine and used for experimentation after 14 days in vitro.

Primary Cerebellar Granule Neurons

Primary cerebellar granule neurons are prepared from dissociated cerebella of 8-day-old Sprague-Dawley rat pups. 2.5×10⁶ cells are plated on polylysine-coated 35-mm culture dish with 2 mL of BME (basal medium Eagle's, GIBCO), supplemented with 10% fetal bovine serum, 25 mM glutamine and incubated at 37° C. in 95% air/5% CO₂ incubator. Ten micromolar cytosine arabinoside (Sigma) is added to the culture medium 18-22 hours after plating to eliminate the proliferation of non-neuronal cells. After 7 or 8 days culturing, the cells are ready for assays.

2. Methods of Inducing Cell Death

Apoptosis Assay

Neurons are pretreated with the ganglioside mimetics for three days (1 to 100 μM in BME containing 25 mM K⁺). They are then rinsed twice in Locke's solution (154 mM NaCl, 5.6 mM KCl, 3.6 mM NaHCO₃, 2.3 mM CaCl₂, 1 mM MgC_{2,} 5.6 mM glucose and 10 mM HEPES, pH 7.4). Apoptosis is then induced by deprivation of serum and depolarizing concentration of K⁺. The neurons are incubated in BME containing 5 mM K⁺in the absence of serum (apoptotic medium) supplemented with 1 μM of (+)-MK-801 (dibezocycloheptenimine, obtained from RBI) to prevent glutamate-induced necrosis.

NMDA Excitotoxicity (See Dawson, et al., Proc. Natl. Acad. Sci. USA 88:7797 (1991)

Stock solutions of 10 mM NMDA and 10 mM glycine in sterile H₂O are diluted to working concentrations of 500 M and 10 M respectively in control salt solution (CSS) without magnesium (120 mM NaCl, 5.4 mM KCl, 1.8 mM CaCl₂, 25 mM Tris-hydrocloride pH 7.4 at room temperature, 15 mM glucose). Cells are pretreated with ganglioside mimetics for 3 days. The complete media is then carefully removed from the cells and gently washed with CSS without magnesium three times. A working solution of NMDA/glycine/CSS is added to the cells for 5 minutes, then promptly aspirated and replaced with CSS containing MgCl₂ (1 mM) to stop the reaction. Cells are then cultured in complete media with and without ganglioside mimetics for another 20-24 hours and then assessed for appropriate incubator for cell survival (trypan blue exclusion and Hoescht/propidium iodide staining detailed below).

1-Methyl-4-Phenyl-1,2,3,6-Tetrahydropyridine (MPP+) Excitotoxcity

For primary ventral mesencephalic cultures, MPP in a range of 1-20 M is added (Research Biochemicals-Sigma) to culture media on culture day 4. Neuronal survival is assessed 24-48 hours later by double immunostaining of anti-TH (Boehringer) and anti-microtubular associated protein 2 (MAP2) (Boehringer).

For SH-SY5Y cells ENRfu (Cassarino et al 2000), cells at 90% confluency are exposed to MPP+ in the range of 0.1-5 mM for 24-48 hours. Neuronal survival is assessed 24-48 hours later by double immunostaining of anti-TH (Boehringer).

III. Measurement of Cell Death

Neuronal cell survival is assayed by Hoesch and Propridium Iodine (PI) counting. The cells are stained with control salt solution (120 mM NaCl, 5.4 mM KCl, 1.8 mM CaCl₂.H₂O, 15 mM Glucose, 25 mM Tris—HCl, 0.8 mM MgCl₂, pH 7.4) with 20 μg/ml Hoesch and 2 μM propidium iodine. The cells are counted by fluorescence microscopy. Both live and dead cells are stained by Hoesh and only the dead cells are stained by PI.

Alternatively, cell death/survival is assessed using a caspase-3 assay. Cells are harvested by washing twice in ice-cold PBS and homogenized by 75 strokes in a Dounce homogenizer using the tight fitting pestle in 100 μL of 0.32 M sucrose in 20 mM Tricine—OH, pH 7.8 (US Biochemicals) supplemented with 30 mM KCl, 5 mM MgCl₂, 1 mM DTT, and 4-(2-aminoethyl)-benzensulfonyl fluroide (ARBSF, Sigma), 10μg/mL aprotinin (Sigma), and 20 μg/mL leupeptin and pepstatin (US Biochemicals). Following centrifugation for 30 minutes at 15,000× g at 4° C., postnuclear supernatants are stored at −80° C. prior to usage. The caspase 3 activation is measured using The FluorAce Apopain Assay kit (Biorad) by adding Z-DEVD-AFC and measuring blue-green Fluorescence intensity.

### EXAMPLE 2

### Immunosupression Assay (See Bruunsgaard, et al., Clin Exp Immunol 119(3):433 (2000)

Peripheral blood monocyte are isolated by Ficoll—Hypague (Pharmacia) density gradient centrifugation from heparinized (50 U/ml) blood (see Boyum, et al., Scand J Clin Lab Invest Suppl. 97:9-29 (1968)). Briefly, heparinized blood is gently laid on top of FICOLL (obtained from Pharmacia) (FICOLL to serum ratio 1:2) and then centrifuged at 1600 rpm for 25 minutes. The buffy coat is aspirated and washed two times and resuspended in RPMI 1640. Cells are cultured in RPMI 1640 with 10% fetal calf serum at the density of 2×10⁵ cells per well in 96 well round-bottom microtiter plates containing 20 mg/ml phytohaemagglutinin for 24 hours with and without ganglioside mimetics (1 to 100 μM). The proliferation of lymphocyte is assay be adding [³H] TDR (1 μCi/well, 5 Ci/mmol) for 18 hours. The plates are then harvested and counted using a scintillation counter.

### EXAMPLE 3

### Neurite Out Growth Assay

Dorsal root ganglia (DRG) neuronal cultures are established from Sprague-Dawley rats at embryonic age of 15 days (Harlan Inc., Indianapolis, Ind.) (see Eldridge, et al., J. Cell. Biol. 105:1023-1034 1987)). Briefly, the embryos are dissected and the spinal cords isolated. The DRGs are then separated from the spinal cords and placed in CMF medium. The DRG neurons are then dissociated with 0.25% trysin and plated into 8-well chamber slides (Nalge Nunc, Chicago, Ill.) that were coated with rat tail collagen (Collaborative Biomedical Products, Bedford, Mass.). Ganglioside is added in various concentrations from 1 μm to 100 μM. The neurite outgrowth is assessed by measuring the length of the neurite after 48 hours.

While this invention has been particularly shown and described with references to preferred embodiments thereof, it will be understood by those skilled in the art that various changes in form and details may be made therein without departing from the scope of the invention encompassed by the appended claims.

What is claimed is:

1. A synthetic ganglioside comprising a deamino-(2-O-substituted)-sphingosine group and pharmaceutically acceptable salts of the synthetic ganglioside.

2. The synthetic ganglioside of claim 1 wherein the deamino-(2-O-substituted)-sphingosine group is represented by the following structural formula: 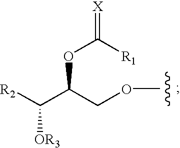
wherein:
X is ═O or —H₂;
R₁ and R₂ are independently a substituted or unsubstituted straight chain or branched hydrocarbyl group, wherein the hydrocarbyl group optionally comprises —S—, —S(O)—, —SO₂—, —O— —NHCO—, —CONH—, —C(O)O—, —OC(O)— or —NR—;
R₃ is —H, —OS(O)₂OH, —OP(O)₂OH, —OP(O)₂OP(O)₂OH, —ON(O)OH; and
each R is independently —H, an aliphatic group, a substituted aliphatic group, an aryl group or a substituted aryl group.

3. The synthetic ganglioside of claim 2 wherein R₁ and R₂ are independently a substituted or unsubstituted straight chain or branched aliphatic group; and R₃ is —H.

4. The synthetic ganglioside of claim 3 wherein R₁ and R₂ are independently a straight chain aliphatic group optionally substituted with one or more groups selected from chloride, bromide, iodide, —OH, —OR, keto, ketal, acetal, ═O, —COR, —N═NR, —SR, —COOR, —SO₃R, —SO₂NRₐR_{b}, —S(O)R, —SO₂R, —CN and —NRₐR_{b}; and
Rₐ and R_{b} are independently —H, an aliphatic group, a substituted aliphatic group, an aryl group or a substituted aryl group, or, taken together with the nitrogen atom to which they are bonded, a C2-C6 substituted or unsubstituted alkylene group.

5. The synthetic ganglioside of claim 3 wherein the synthetic ganglioside is represented by the structural formula A-B, wherein:
A is the deamino-(2-O-substituted)-sphingosine group; and
B is an oligosaccharide substituted with between one and about five sialic acid residues.

6. The synthetic ganglioside of claim 5 wherein the oligosaccharide has up to four monosaccharides or derivatives thereof.

7. The synthetic ganglioside of claim 6 wherein B is the sialic acid substituted oligosaccharide of the ganglioside GM1, GM2, GD1b, GT1b, GD2, GM3 and GD3.

8. The synthetic ganglioside of claim 7 wherein:
R₁ and R₂ are independently a straight chained aliphatic group optionally substituted one or more halide groups.

9. The synthetic ganglioside of claim 6 wherein:
R₁ and R₂ are independently a straight chained aliphatic group optionally substituted one or more halide groups.

10. The synthetic ganglioside of claim 8 wherein:
R₁ is a straight chain C1-C24 alkyl group optionally substituted with one or more halide groups;
R₂ is —CH₂ CH₂—(CH₂)ₙCH₃ or trans-CH═CH—(CH₂)ₙCH₃; and
n is an integer from about nine to about twenty-one.

11. The synthetic ganglioside of claim 10 wherein:
R₁ is a C1-C2 alkyl group optionally substituted with one, two or three chloride groups; and
n is an integer from about ten to about fourteen.

12. The synthetic ganglioside of claim 11 wherein B is the sialic acid substituted oligosaccharide of the ganglioside GM1.

13. The synthetic ganglioside of claim 12 wherein R₁ is —CH₂CHCl₂ and R₂ is trans-CH═CH(CH₂)₁₂CH₃.

14. The synthetic ganglioside of claim 8 wherein:
R₁ is a straight chain C12-C24 alkyl group; and
R₂ is a straight chain C1-C24 alkyl group optionally substituted with one or more halide groups.

15. The synthetic ganglioside of claim 14 wherein:
R₁ is a straight chain C13-C17 alkyl group; and
R₂ is a C1-C2 alkyl group optionally substituted with one, two or three chloride groups.

16. The synthetic ganglioside of claim 15 wherein B is the sialic acid substituted oligosaccharide of the ganglioside GM1.

17. The synthetic ganglioside of claim 16 wherein R₁ is —(CH₂)₁₄CH₃ and R₂ is —CHCl₂.

18. The synthetic ganglioside of claim 8 wherein:
R₁ and R₂ are independently a straight chain C1-C24 alkyl group substituted with one or more halide groups.

19. The synthetic ganglioside of claim 18 wherein B is the sialic acid substituted oligosaccharide of the ganglioside GM.

20. The synthetic ganglioside of claim 19 wherein at least one of R₁ or R₂ is —CHCl₂.

21. A method of treating a subject with a neurological disease or condition comprising administering to the subject an effective amount of the synthetic ganglioside of claim 1.

22. The method of claim 21 wherein the subject is in need of treatment with a neuroprotective agent.

23. The method of claim 21 wherein the subject is in need of treatment with a neuritogenic agent.

24. The method of claim 21 wherein the subject is in need of treatment with a neurogenic agent.

25. The method of claim 21 wherein the subject is in need of treatment for ischemia, hypoxia, epilepsy, metabolic dysfunction, aging, toxic diseases, Alzheimer's disease, Amytropic Lateral Sclerosis, Parkinson's disease, Huntington's chorea, stroke, transverse myelitis, neuronal damage, spinal cord injuries or neuropathies associated with diabetes.

26. The method of claim 21 wherein the deamino-(2-O-substituted)-sphingosine group is represented by the following structural formula: 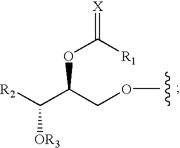
wherein:
X is ═O or —H₂;
R₁ and R₂ are independently a substituted or unsubstituted straight chain or branched hydrocarbyl group, wherein the hydrocarbyl group optionally comprises —S—, —S(O)—, —SO₂—, —O— —NHCO—, —CONH—, —C(O)O—, —OC(O)— or —NR—;
R₃ is —H, —OS(O)₂OH, —OP(O)₂OH, —OP(O)₂OP(O)₂OH, —ON(O)OH; and
each R is independently —H, an aliphatic group, a substituted aliphatic group, an aryl group or a substituted aryl group.

27. The method of claim 26 wherein R₁ and R₂ are independently a substituted or unsubstituted straight chain or branched aliphatic group; and R₃ is —H.

28. The method of claim 27 wherein R₁ and R₂ are independently a straight chain aliphatic group optionally substituted with one or more groups selected from chloride, bromide, iodide, —OH, —OR, keto, ketal, acetal, ═O, —COR, —N═NR, —SR, —COOR, —SO₃R, —SO₂NRₐR_{b}, —S(O)R, —SO₂R, —CN and —NRₐR_{b}; and
Rₐ and R_{b} are independently —H, an aliphatic group, a substituted aliphatic group, an aryl group or a substituted aryl group, or, taken together with the nitrogen atom to which they are bonded, a C2-C6 substituted or unsubstituted alkylene group.

29. The method of claim 27 wherein the synthetic ganglioside is represented by the structural formula A-B, wherein:
A is the deamino-(2-O-substituted)-sphingosine group; and
B is an oligosaccharide substituted with between one and about five sialic acid residues.

30. The method of claim 29 wherein the oligosaccharide has up to four monosaccharides or derivatives thereof.

31. The method of claim 30 wherein B is the sialic acid substituted oligosaccharide of the ganglioside GM1, GM2, GD1b, GT1b, GD2, GM3 and GD3.

32. The method of claim 31 wherein:
R₁ and R₂ are independently a straight chained aliphatic group optionally substituted one or more halide groups.

33. The method of claim 30 wherein:
R₁ and R₂ are independently a straight chained aliphatic group optionally substituted one or more halide groups.

34. The method of claim 33 wherein:
R₁ is a straight chain C1-C24 alkyl group optionally substituted with one or more halide groups;
R₂ is —CH₂ CH₂—(CH₂)ₙCH₃ or trans-CH═CH—(CH₂)ₙCH₃; and
n is an integer from about nine to about twenty-one.

35. The method of claim 34 wherein:
R₁ is a C1-C2 alkyl group optionally substituted with one, two or three chloride groups; and
n is an integer from about ten to about fourteen.

36. The method of claim 35 wherein B is the sialic acid substituted oligosaccharide of the ganglioside GM1.

37. The method of claim 36 wherein R₁ is —CH₂CHCl₂ and R₂ is trans-CH═CH(CH₂)₁₂CH₃.

38. The method of claim 32 wherein:
R₁ is a straight chain C12-C24 alkyl group; and
R₂ is a straight chain C1-C24 alkyl group optionally substituted with one or more halide groups.

39. The method of claim 38 wherein:
R₁ is a straight chain C13-C17 alkyl group; and
R₂ is a C1-C2 alkyl group optionally substituted with one, two or three chloride groups.

40. The method of claim 39 wherein B is the sialic acid substituted oligosaccharide of the ganglioside GM1.

41. The method of claim 40 wherein R₁ is —(CH₂)₁₄CH₃ and R₂ is —CHCl₂.

42. The method of claim 32 wherein:
R₁ and R₂ are independently a straight chain C1-C24 alkyl group substituted with one or more halide groups.

43. The method of claim 42 wherein B is the sialic acid substituted oligosaccharide of the ganglioside GM1.

44. The method of claim 43 wherein at least one of R₁ or R₂ is —CHCl₂.

45. A method of treating a subject in need of immune system suppression, said method comprising the step of administering an effective amount of the synthetic ganglioside of claim 1.

46. The method of claim 45 wherein the subject is an organ, bone marrow or stem cell transplant recipient.

47. The method of claim 45 wherein the subject is in need of treatment for multiple sclerosis, rheumatoid arthritis, paraneoplastic diseases, sarcoid, chronic polyarthritis, lupus erythematosus, juvenile-onset diabetes mellitus, Sjögren, psoriasis, Scleroderma or vasculitides.

48. The method of claim 45 wherein the deamino-(2-O-substituted)-sphingosine group is represented by the following structural formula: 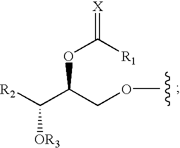
wherein:
X is ═O or —H₂;
R₁ and R₂ are independently a substituted or unsubstituted straight chain or branched hydrocarbyl group, wherein the hydrocarbyl group optionally comprises —S—, —S(O)—, —SO₂—, —O— —NHCO—, —CONH—, —C(O)O—, —OC(O)— or —NR—;
R₃ is —H, —OS(O)₂OH, —OP(O)₂OH, —OP(O)₂OP(O)₂OH, —ON(O)OH; and
each R is independently —H, an aliphatic group, a substituted aliphatic group, an aryl group or a substituted aryl group.

49. The method of claim 22 wherein R₁ and R₂ are independently a substituted or unsubstituted straight chain or branched aliphatic group; and R₃ is —H.

50. The method of claim 49 wherein R₁ and R₂ are independently a straight chain aliphatic group optionally substituted with one or more groups selected from chloride, bromide, iodide, —OH, —OR, keto, ketal, acetal, ═O, —COR, —N═NR, —SR, —COOR, —SO₃R, —SO₂NRₐR_{b}, —S(O)R, —SO₂R, —CN and —NRₐR_{b}; and
Rₐ and R_{b} are independently —H, an aliphatic group, a substituted aliphatic group, an aryl group or a substituted aryl group, or, taken together with the nitrogen atom to which they are bonded, a C2-C6 substituted or unsubstituted alkylene group.

51. The method of claim 49 wherein the synthetic ganglioside is represented by the structural formula A-B, wherein:
A is the deamino-(2-O-substituted)-sphingosine group; and
B is an oligosaccharide substituted with between one and about five sialic acid residues.

52. The method of claim 51 wherein the oligosaccharide has up to four monosaccharides or derivatives thereof.

53. The method of claim 52 wherein B is the sialic acid substituted oligosaccharide of the ganglioside GM1, GM2, GD1b, GT1b, GD2, GM3 and GD3.

54. The method of claim 53 wherein:
R₁ and R₂ are independently a straight chained aliphatic group optionally substituted one or more halide groups.

55. The method of claim 52 wherein:
R₁ and R₂ are independently a straight chained aliphatic group optionally substituted one or more halide groups.

56. The method of claim 55 wherein:
R₁ is a straight chain C1-C24 alkyl group optionally substituted with one or more halide groups;
R₂ is —CH₂ CH₂—(CH₂),CH₃ or trans-CH═CH—(CH₂),CH₃; and
n is an integer from about nine to about twenty-one.

57. The method of claim 56 wherein:
R₁ is a C1-C2 alkyl group optionally substituted with one, two or three chloride groups; and
n is an integer from about ten to about fourteen.

58. The method of claim 57 wherein B is the sialic acid substituted oligosaccharide of the ganglioside GM1.

59. The method of claim 58 wherein R₁ is —CH₂CHCl₂ and R₂ is trans-CH═CH(CH₂)₁₂CH₃.

60. The method of claim 54 wherein:
R₁ is a straight chain C12-C24 alkyl group; and
R₂ is a straight chain C1-C24 alkyl group optionally substituted with one or more halide groups.

61. The method of claim 60 wherein:
R₁ is a straight chain C13-C17 alkyl group; and
R₂ is a C1-C2 alkyl group optionally substituted with one, two or three chloride groups.

62. The method of claim 61 wherein B is the sialic acid substituted oligosaccharide of the ganglioside GM1.

63. The method of claim 62 wherein R₁ is —(CH₂)₁₄CH₃ and R₂ is —CHCl₂.

64. The method of claim 54 wherein:
R₁ and R₂ are independently a straight chain C1-C24 alkyl group substituted with one or more halide groups.

65. The method of claim 64 wherein B is the sialic acid substituted oligosaccharide of the ganglioside GM.

66. The method of claim 65 wherein at least one of R₁ or R₂ is —CHCl₂.

67. A synthetic ganglioside comprising a modified sphingosine represented by the following structural formula: 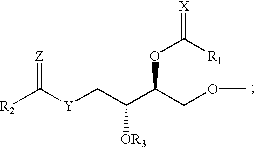
wherein:
X is ═O or —H₂;
Y is —O— or —NH—;
Z is ═O or —H₂;
R₁ and R₂ are independently a substituted or unsubstituted straight chain or branched hydrocarbyl group, wherein the hydrocarbyl group optionally comprises —S—, —S(O)—, —SO₂—, —O— —NHCO—, —CONH—, —C(O)O—, —OC(O)— or —NR—;
R₃ is —H, —OS(O)₂OH, —OP(O)₂OH, —OP(O)₂OP(O)₂OH, —ON(O)OH; and
each R is independently —H, an aliphatic group, a substituted aliphatic group, an aryl group or a substituted aryl group.

68. The synthetic ganglioside of claim 67 wherein Y is or —NH— and R₃ is —H.

69. A method of treating a subject with a neurological disease or condition comprising administering to the subject an effective amount of the synthetic ganglioside of claim 67.

70. A method of treating a subject in need of immune system suppression, said method comprising the step of administering an effective amount of the synthetic ganglioside of claim 67.
